(19) United States
(12) Patent Application Publication
(10) Pub. No.: US 2022/0330926 A1
(43) Pub. Date: Oct. 20, 2022

(54) BIOPSY DEVICE

(71) Applicant: Early Bird Medical, Inc., Malvern, PA (US)

(72) Inventors: Jason KEISER, Exton, PA (US); E. Skott GREENHALGH, Gladwyne, PA (US); Michael DOTSEY, Chester Springs, PA (US)

(21) Appl. No.: 17/722,262

(22) Filed: Apr. 15, 2022

Related U.S. Application Data
(60) Provisional application No. 63/176,174, filed on Apr. 16, 2021.

(51) Int. Cl. A61B 10/02; A61B 10/00

(52) U.S. Cl. CPC A61B 10/02; A61B 10/0045; A61B 2010/0074; A61B 2010/0216; A61B 2010/0208

(57) ABSTRACT

Apparatuses and methods for biopsying body vessels having small lumen, such as Fallopian tubes. In particular, described herein are apparatuses (e.g., systems and devices) and methods that include an outer member having a stepped distal end region, a flexible inner shaft, and a biopsy collector, and methods of using them.

### PRIORITY CLAIM

This patent application claims priority to U.S. Provisional Patent Application No. 63/176,174, titled “BIOPSY DEVICE,” filed on Apr. 16, 2021, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are apparatuses and methods for performing biopsies on lumina, including small lumina, such as Fallopian tubes. In particular, described herein are apparatuses (e.g., systems and devices) and methods that evert a textile to sample tissue from a vessel, conduit, or other lumina such as the fallopian tubes without applying substantial shear force.

### BACKGROUND

A biopsy is the removal of tissue in order to examine the tissue for disease. Biopsies are frequently used to diagnose cancer, but they can also help identify other conditions such as infections and inflammatory and autoimmune disorders. Biopsies may also be performed to match organ tissue before a transplant and to look for signs of organ rejection following a transplant. Biopsies are performed in many areas of the body and for many reasons. Further, the tissue samples can be taken from any part of the body, including, but not limited to, small channels, ducts, and tubes such as (but not limited to) the fallopian tubes (e.g., uterine tubes).

Performing biopsies within small lumina may be difficult due not only to their associated small diameters, but also due to the lumina surface irregularities and undulations. In some cases, small lumina may restrict or block an insertion and/or deployment of a biopsy tool.

Thus, there exists a need for devices and processes to allow samples to be obtained from small body ducts and tubes, such as the fallopian tubes, for evaluation of tissue without the need for a skin incision.

### SUMMARY OF THE DISCLOSURE

Described herein are biopsy apparatuses configured to take biopsy (e.g., tissue, cell, etc.) samples from within a small diameter lumen of a body. These biopsy apparatuses include devices, which may be referred to herein as biopsy devices, and systems including them. These apparatuses may gently remove biopsy samples from the wall(s) of a vessel lumen without risk of rupture of the vessel and with minimal irritation of the vessel.

In general, these apparatuses may include an outer tubular member, which may have a stepped (in some examples tapered) distal end region, a flexible inner shaft, and a biopsy collector. The biopsy collector may be formed from a compliant, a woven, knitted or braided material that everts over itself to extend from the distal end of the device and contact the tissue within the lumen. The flexible inner shaft may be axially and concentrically arranged with respect to the outer tubular member. The biopsy collector may be anchored to the flexible inner shaft and the distal end region (e.g., the stepped distal end region) of the outer tubular member. In some examples a continuous force may be exerted on the flexible inner shaft. The biopsy collector may be deployed from a distal end of the outer tubular member. As the biopsy collector is deployed into the lumen, the biopsy collector can contact walls of the lumen and collect cells and/or tissue. The biopsy collector may be withdrawn back into the outer tubular member.

In general, the biopsy devices may include a handle configured to support the outer tubular member, the flexible inner shaft, and (in some examples) a bias, such as a spring, configured to exert a continuous force on the inner shaft, which may advance (or help advance) the inner shaft to deploy the biopsy collector. In some examples the handle may include a thumb slide to move the flexible inner shaft relative to the outer tubular member.

Also described herein are methods of collecting biopsy material from within a lumen. The method can include guiding an outer tubular member of a biopsy device to the lumen to be biopsied, while restraining a deployment bias (e.g. spring) in a locked configuration. The stepped distal end region of the biopsy device may be advanced into a narrow or tortious region of the anatomy so that a distal end of the device is positioned within the lumen to be biopsied. In some examples, the stepped distal end region is sized (e.g., having a length of between 2 cm to 5 cm) to allow the distal end region to extend into the lumen beyond a constricted region. Once the distal end region is positioned within the region to be biopsied, the restraint (e.g., lock) on the deployment bias may be released, to advance the flexible inner shaft thereby extending the biopsy collector distally into the lumen.

In some examples, the bias may apply a distal force to the biopsy collector by driving the flexible inner member (“flexible inner shaft”). The biopsy collector may be anchored to the flexible inner shaft and an outer surface of the stepped distal end region. The flexible inner shaft may be slidably coupled within the outer tubular member.

In general, even in examples in which a bias is not used to deploy the biopsy collector, such as when the biopsy collector is manually deployed, the apparatus may include an inner internal support wire or microcatheter that may provide internal support to the biopsy collector, to prevent it from buckling, kinking or collapsing inward as it is deployed distally, e.g., by advancing the flexible inner shaft. The support may be fixed within the flexible inner shaft, or it may be slidable distally/proximally within the flexible inner shaft. In one example the support may extend from the distal end of the flexible inner shaft (and may be integral with the flexible inner shaft). The biopsy collection device may slide and/or stack up against the outer surface of the support as it is advanced distally.

In some examples the method may further include retracting the biopsy collection device; for example, by applying a retracting force to the flexible inner shaft to retract the biopsy collector proximally from out of the lumen. The retracting force may be applied automatically or manually.

In some examples the biopsy device may include a stepped distal end region at the distal end of the outer member. Either or both the stepped distal end region and/or the flexible inner shaft may be configured to have a high degree of flexibility and a high column strength, e.g. by including a region with a plurality of cut-outs (e.g., windows). These cut-outs may be oriented in a radial direction and may resist tensile stretch and provide high compressive column strength.

The flexible inner shaft may be arranged within the outer tubular member (including within the stepped distal end region of the outer tubular member), such that the flexible inner shaft may move axially in a distal and proximal direction relative to the stepped distal end region. The biopsy collector may be anchored to the stepped distal end region (e.g., the distal end face and/or an outer surface of the biopsy collector).

In some examples, the biopsy device may include a thumb slide configured to selectively apply a force to advance and/or retract the flexible inner shaft relative to the outer tubular member. Thus, any of these lumen biopsy devices may also include a handle configured to enable a user to operate the thumb slide. In some examples, the handle may include a trigger control to release the deployment bias and deploy the biopsy collector.

The biopsy collector may be configured to evert from the stepped distal end region based at least in part on motion of the flexible inner shaft. The biopsy collector may be configured to evert about the stepped distal end region. The stepped distal end region is configured to exert a continuous force to the biopsy collector, securing the proximal end as the distal end (attached to the flexible inner member) is advanced distally, causing the un-inverted portion, which is the inner portion of the biopsy collector) to compress so the everted distal face advances distally while inverting over itself. As mentioned, the biopsy collector may be a braided, woven or knit material, or a combination thereof. As mentioned, in some of these apparatuses an inner support within the center of the biopsy collector may provide lateral support as it is deployed, so that it does not buckle.

Any of the apparatuses described herein may also include a laterally expanding element that may expand laterally when the apparatus is deployed. In some examples a laterally expanding mesh (e.g., braided, woven, or knit material) may be deployed to expand within the lumen. In some examples the apparatus may include a sleeve at the distal end, e.g. coupled to an end of the biopsy collector. As the biopsy collector is deployed the sleeve portion may be everted and deployed first and may be biased to expand within the lumen to enhance deployment of the biopsy collector and/or contact with the tissue to be biopsied.

In some examples the lumen biopsy devices that may be configured so that the stepped distal end region is coaxially arranged within the outer member (as part of a separate catheter, for example, such as a middle or intermediate catheter) that can be advanced or retracted relative to the outer member. Thus, the stepped distal end region may move axially in a distal and proximal direction relative to the outer member and the middle or intermediate catheter forming the stepped distal end region. The flexible inner shaft may be arranged within the stepped distal end region, such that the flexible inner shaft may more axially in a distal and proximal direction relative to the stepped distal end region to deploy the biopsy collector that is anchored to the stepped distal end region and also to the flexible inner shaft. As mentioned, any of these apparatuses may also include a deployment bias (e.g., a spring) coupled to the stepped distal end region and/or to the flexible inner member and configured to be released to deploy the biopsy collector. Any of these devices may include an inner support to prevent buckling of the biopsy collector. Any of these apparatuses may include a handle including a release for releasing (and/or reloading/re-engaging) the deployment bias. Alternatively or additionally, the handle may include a control (e.g., a slider such as a thumb slide) configured to move the flexible inner shaft proximally (to retract) and/or distally (to advance) relative to the outer tubular member.

Although the methods and apparatuses (including systems and devices) described herein are directed in particular for biopsying the fallopian tubes, any of these devices and methods may be adapted for sampling other region, and particular tubular, body regions, such as the mouth, nasal passages, ear canals, esophagus, brachial passages, rectum, small intestine, etc.

In general, the biopsy devices described herein may not be stepped or tapered on the outer tubular member but may include any of the other features. For example, a biopsy device may include: an outer tubular member having an elongate catheter body and a distal end region; a flexible inner shaft arranged coaxially extending within the outer tubular member; and a biopsy collector having an everted tubular body, the biopsy collector coupled at a first end to a first distal end region to the flexible inner shaft, wherein the biopsy collector everts over itself at a distal face so that a second end of the tubular biopsy collector is coupled to the stepped distal end region end; wherein the flexible inner shaft is configured to move axially within the outer tubular member to drive the distal face of the biopsy collector distally as the flexible inner shaft is moved distally relative to the outer tubular member.

For example, described herein are biopsy devices for taking a biopsy of a lumen, the device comprising: an outer tubular member having an elongate body and a stepped distal end region, wherein the stepped distal end region has an outer diameter that is between 50% to 80% of an outer diameter of the elongate body and extends between 2-5 cm; a flexible inner shaft arranged coaxially and extending within the outer tubular member; and a biopsy collector having an everted tubular body, the biopsy collector coupled at a first end to a first distal end region to the flexible inner shaft, wherein the biopsy collector everts over itself at a distal face so that a second end of the tubular biopsy collector is coupled to the stepped distal end region end; wherein the flexible inner shaft is configured to move axially within the outer tubular member to drive the distal face of the biopsy collector distally as the flexible inner shaft is moved distally relative to the outer tubular member.

Any of these biopsy devices may include a column support extending within a lumen of the biopsy collector (e.g., a nitinol or stainless steel column support, which may be solid), and wherein the biopsy collector is configured to support against the outer surface of the column support as the flexible inner shaft is driven distally. For example, the column support may comprise a rod that is configured to slide axially within a lumen of the flexible inner shaft. Alternatively, the column support may be configured to be fixed relative to the flexible inner shaft.

In any of these apparatuses (e.g., devices, systems, etc.) the biopsy collector may be held within the flexible inner shaft (which may also be referred to herein as a flexible inner member, pusher, or puller). For example, the biopsy collector may be gathered within the lumen of the flexible inner shaft at a distal end, where it may be held in a constrained, collapsed configured to until it is pulled out of the distal end of the flexible inner shaft during deployment. One end of the biopsy collector may be fixed within the lumen of the flexible inner shaft (and/or in some examples to the support); the other end may be coupled to the outer member.

Any of these apparatuses may include a handle at a proximal end of the outer tubular member, and (optionally) a deployment bias coupled to the flexible inner shaft and configured to drive the flexible inner shaft distally to deploy the biopsy collector distally when a lock restraining the deployment bias is released. Any of these handles may include a control, such as a thumb slide that is configured to move the flexible inner shaft relative to the outer tubular member.

In any of these apparatuses, the stepped distal end region may be coaxially arranged within an outer tubular member, such that the stepped distal end region may move axially in a distal and proximal direction relative to the outer tubular member.

Any of these apparatuses may include a sleeve configured to expand radially from a distal end of the device when the biopsy collector is advanced distally. In some examples the biopsy collector comprises a plurality of knitted strands. In some examples the biopsy collector comprises a braid, a knit or a weave.

The stepped distal end may comprise a flexible region comprising a plurality of cut-out regions radially arranged around a circumference of the stepped distal end that are configured to increase flexibility without reducing compressive column strength.

For example, a biopsy device for taking a biopsy of a lumen may include: an outer tubular member having an elongate body and a stepped distal end region; a flexible inner shaft arranged coaxially within the outer tubular member; a biopsy collector having an everted tubular body, the biopsy collector coupled at a first end to the flexible inner shaft and a second end to the outer tubular member, wherein, in an undeployed state, the biopsy collector is longitudinally compressed within a lumen of the flexible inner shaft, further wherein the biopsy collect everts over itself at a distal face; and a column support within the flexible inner shaft; wherein the flexible inner shaft is configured to move axially distally within the outer tubular member to deploy the biopsy collection distally from the outer tubular member so that it everts over itself as the flexible inner shaft is moved distally relative to the outer tubular member.

In some examples the biopsy device for taking a biopsy of a lumen includes: an outer tubular member having an elongate body and a stepped distal end region, wherein the stepped distal end region has an outer diameter that is between 50% to 80% of the outer diameter of the elongate body and wherein the stepped distal end region extends between 2 cm and 5 cm; a flexible inner shaft arranged coaxially extending within the outer tubular member; a biopsy collector having an everted tubular body, the biopsy collector coupled at a first end to the flexible inner shaft, wherein the biopsy collect everts over itself at a distal face so that a second end of the tubular biopsy collector is coupled to the stepped distal end region end; and a column support within the biopsy collector and/or the flexible inner shaft; wherein the flexible inner shaft is configured to move axially distally within the outer tubular member to drive the distal face of the biopsy collector distally as the flexible inner shaft is moved distally relative to the outer tubular member.

Also described herein are methods of collecting biopsy material from within a lumen, the method comprising: guiding an outer tubular member of a biopsy device to a lumen; advancing a stepped distal end region of the outer tubular member into the lumen until a shoulder region of the outer tubular member is against or adjacent to an opening into the lumen or a bend in the lumen; advancing a flexible inner shaft of the device distally relative to the outer tubular member to evert a tubular and flexible biopsy collector over itself as the biopsy collector extends distally, wherein a distal face of the biopsy collector advances distally as the flexible inner shaft is advanced distally; collecting biopsy material onto an inverted outer face of the biopsy collector within the lumen; and retracting the flexible inner shaft proximally to invert the inverted outer face of the biopsy collector back into the outer tubular member. Advancing the flexible inner shaft of the device distally relative to the outer tubular member to evert the biopsy collector over itself may comprise pulling the biopsy collector out of the flexible inner shaft. In some examples, advancing the flexible inner shaft comprises releasing a lock to automatically advance the flexible inner shaft of the device distally relative to the outer tubular member. For example, releasing may comprise releasing a deployment bias.

Any of these methods may include supporting the tubular and flexible biopsy collector on a column support within the tubular and flexible biopsy collector as the flexible inner shaft is advanced.

The method may include expanding a sleeve radially from a distal end of the outer tubular member while advancing the flexible inner shaft of the device distally. In some examples, advancing the flexible inner shaft comprises operating a thumb slide on a handle of the biopsy device. Guiding the outer tubular member of the biopsy device to the lumen may comprise positioning the outer tubular member at the opening to a fallopian tube.

For example, a method of collecting biopsy material from within a lumen may include: guiding an outer tubular member of a biopsy device to a lumen; advancing a stepped distal end region of the outer tubular member into the lumen until a shoulder region of the outer tubular member is positioned against an opening into the lumen or a bend in the lumen; advancing a flexible inner shaft of the device distally relative to the outer tubular member to evert a tubular and flexible biopsy collector over itself as the biopsy collector extends out of the flexible inner shaft, wherein a distal face of the biopsy collector advances distally as the flexible inner shaft is advanced distally; collecting biopsy material onto an inverted outer face of the biopsy collector within the lumen; and retracting the flexible inner shaft proximally to invert the inverted outer face of the biopsy collector back into the outer tubular member.

All of the methods and apparatuses described herein, in any combination, are herein contemplated and can be used to achieve the benefits as described herein.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A shows an example of a biopsy apparatus.

FIG. 1B shows an example of a biopsy apparatus.

FIG. 2A shows an example of an inner/outer member assembly, as described herein.

FIG. 2B shows one example of a kink-resistant catheter, including a support that may be used with any of the biopsy devices described herein.

FIG. 2C is another example of a portion of an apparatus, including a highly flexible distal tip region that is configured to have a high column force.

FIG. 2D schematically illustrates an enlarged view of the distal tip region from FIG. 2C.

FIG. 3A shows on example of a catheter shaft assembly that may form at least a portion an outer assembly of an apparatus as described herein.

FIG. 3B shows another example of a catheter shaft assembly, as described herein.

FIG. 3C shows an example of an apparatus including an internal inverting member column support element that improves kink resistance at the distal end region of the apparatus.

FIG. 4 shows a biopsy collector portion of an apparatus as described herein.

FIG. 5 shows another example of a biopsy apparatus, as described herein.

FIG. 6 shows another example of a biopsy apparatus, as described herein.

FIG. 7 is a flowchart depicting an example of one method for performing a luminal biopsy using a biopsy device.

FIG. 8 shows an example of an apparatus including an outwardly-expanding everting braid that may assist in deploying the apparatus.

FIGS. 9A-9B illustrate examples of apparatuses including a self-sliding sheath to assist in deploying the apparatus.

FIGS. 10A-10B illustrate one example of an apparatus deploying the biopsy collector in incremental (ratcheting) steps (FIG. 10A) and retracting the biopsy collector in a single action (FIG. 10B).

FIGS. 11A-11B illustrate one example of an apparatus that is configured to bias advancement/deployment of the biopsy collector distally.

### DETAILED DESCRIPTION

Described herein are methods and apparatuses (e.g., devices, systems, tools, etc.) for taking or collecting a biopsy from the inside of a small tube (e.g., lumen) without rupturing or dissecting the small tube. The methods and apparatuses described herein may be used for any body lumen, including in particular small-diameter body lumina, such as the fallopian tubes, urethra, etc.

In general, these apparatuses (referred to herein as biopsy devices) may include an outer tubular member (e.g., which may be optionally referred to as an outer catheter or an outer member). The distal end of the outer tubular member may be adapted to have a stepped distal end region so that the outer diameter of the outer tubular member transitions from a first outer diameter (OD) to a second, smaller OD (e.g., between 85%-50% of the larger OD). This transition may be abrupt (e.g., occurring over less than 1-4 mm) or it may be gradual (typically referred to herein as a taper). In particular, the transition may be over a length of 2 mm or less (1.5 mm or less, 1 mm or less, 0.75 mm or less, 0.5 mm or less, etc.) so that a shoulder region is formed. This shoulder region may assist in deploying these devices within the target lumen, particularly where the body lumen to be biopsied includes a constriction, such as a narrowing (formed by a stricture, a sphincter, etc.) and/or a bend in the channel of, or leading into, the lumen to be biopsied. The stepped distal region may therefore match the anatomy of the lumen. The length of the stepped distal end region may be configured to match the anatomy to be sampled. In some examples the stepped distal end region has a length that is between about 1 cm and about 5 cm long (e.g., between about 1.5 cm and 4 cm, between about 2 cm and 4 cm, between about 2.5 cm and 4 cm, etc.). The stepped distal end region may be integrally formed at the distal end of the outer tubular member and may be considered a part of the outer tubular member, or it may be formed separately, e.g., as catheter attached to the distal end region. Optionally, in some examples the stepped distal end region may be formed of an intermediate catheter that can be separately advanced/retracted to adjust the length of the stepped distal end region. In some examples the stepped distal end region is fixed relative to the distal end of the outer tubular member. The shoulder region has been found to be particularly helpful in navigating and taking a biopsy within the fallopian tubes. In particular the shoulder region may be configured so that the apparatus may be immediately and conveniently positioned using the shoulder region near the initial bend in the fallopian tube.

Any of these devices may include a flexible inner shaft (which may alternatively be referred to as an inner catheter, deployment catheter, pusher, or deployment pusher) that is coaxially arranged and slidable within the outer catheter (and stepped distal end region). The flexible inner shaft may extend within the length of the outer tubular member. The flexible inner shaft may be a catheter, having an open or partially open, central lumen, or it may be a solid rod or wire of material. In some examples the pusher may be a hollow microcatheter. The flexible inner shaft (“pusher” or “puller”) may be formed of any appropriate material, e.g., stainless steel, spring steel, Nitinol, a polymer, etc.

Any of these devices may also include a biopsy collector. The biopsy collector may be a woven, braided or knit material formed of one or more strands of material, and may be generally flexible and configured to evert over itself, as an everting tube. A first end (referred to herein as the distal end) may be coupled to a distal end region of the flexile inner shaft (“pusher” or “puller”); the second end (referred to herein as a proximal end) may be coupled to the outer tubular member, and in particular, to the stepped distal end region. In some examples described herein the second end of the biopsy collector may be coupled to a sleeve region (or the proximal end of the biopsy collector may itself be configured as a sleeve region) that may then couple to the outer tubular member and/or stepped distal end region.

As mentioned, the stepped distal end region may be integral with, coupled to, and in some cases coaxially arranged with respect to, the outer member. The stepped distal end region may be rigidly coupled to the outer tubular member. Alternatively in some examples the stepped distal end region may be movable (e.g., a sliding, rotating, or other non-fixed attachment) relative to the outer tubular member.

As mentioned, the flexible inner shaft may be slidably disposed within the outer tubular member and the stepped distal end region. The flexile inner shaft may be referred to herein as a pusher or puller, and may be a wire, microcatheter, etc. The biopsy collector may have a first end coupled to the flexible inner shaft and a second end coupled to the stepped distal end region. In some examples the biopsy collector (e.g., a proximal end of the biopsy collector), or a sleeve to which the biopsy collector is attached, or a proximal end region of the biopsy collector itself, may be expandable, and/or may be biased to expand radially when deployed within the so that it may expand against the walls of the body lumen from which the biopsy is being taken.

The biopsy collector may be braided, woven or knitted (knit), as described herein, and may also be configured to remove material (biopsy material) from the lumen wall without puncturing or otherwise rupturing the lumen wall but may also be configured to retain tissue to be removed. The biopsy collector may be formed of filaments (strands) of material that are woven, knit and/or braided. The filament may be formed of a natural or synthetic material, including polymeric materials, and metallic materials. These materials may be configured to expand (e.g., self-expand) upon release from the device, as will be described in greater detail here, and may brush or scrape against much of the surface of the (often uneven, wrinkled or tortious) surfaces forming the lumen, without damaging or risking penetration of the lumen.

### Rolling Tube Biopsy Device

The biopsy collector everts over itself distally to sample the inner wall surface of a body vessel or lumen. The everting biopsy collector may gently roll out into a small vessel or conduit to form a torus shape at a distalmost region as it everts over itself. The outer walls of the torus-shaped collector (as it everts) may contact the wall(s) of the vessel to collect material (cells, tissue, mucus, etc.) from them. In particular, the device may be stepwise or ratcheted out from the device when deploying. The biopsy collector may be biased to provide a quick, ratcheted release. When the biopsy collector is retracted back into the biopsy device, e.g., by pulling the inner flexible member proximally, inverting the biopsy collector and pulling it back into the biopsy device (e.g., within the inner lumen of the biopsy device and eventually within the inner lumen of the outer tubular member and/or stepped distal end region), the biopsy material, such as tissue and/or cells, mucus, etc., captured by the biopsy collector may be protected. The rolling and everting motion of the biopsy collector may reduce or eliminate deployment shear force to the vessel/conduit wall. The examples described herein may therefore avoid hydraulic forces.

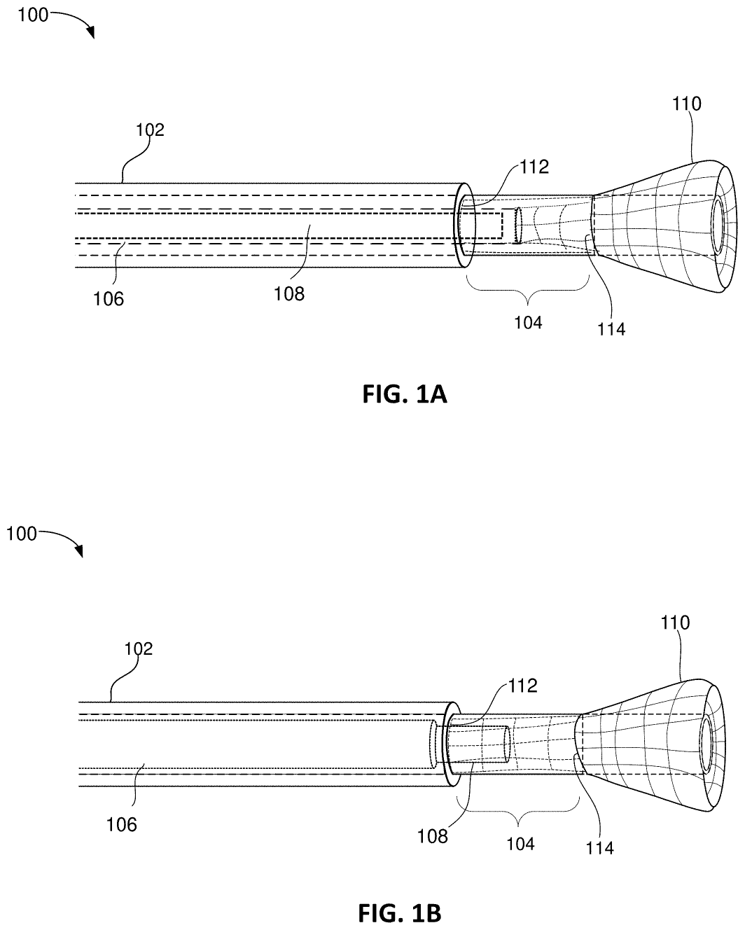

FIG. 1A shows one example of a biopsy device 100, as described herein, shown partially deployed. The biopsy device 100 may include an outer member 102 (having a stepped distal end region 104), a flexible inner shaft 106, an optional column support 108, and a biopsy collector 110. Illustrations of the outer member 102, including the stepped distal end region 104, the flexible inner shaft 106, the column support 108, and the biopsy collector 110 in FIG. 1A may not be to scale. The flexible inner shaft 106 may be axially and concentrically disposed with respect to the outer member 102, including the stepped distal end region 104. The stepped distal end region 104 may be axially and concentrically disposed with respect to the outer member 102. In some examples, the stepped distal end region 104 may be formed of an intermediate catheter member that is longer than the outer member 102. The outer tubular member 102 and the flexible inner shaft 106, may be formed from any technically feasible structure and material. For example, the outer tubular member and/or the flexible inner member may be a catheter, coil, or the like. In some examples, the stepped distal end region 104, the outer member 102, and the flexible inner shaft 106 may be individually and collectively highly flexible, enabling biopsy collection through uneven, irregular, and/or undulating lumina.

The column support 108 in FIG. 1A is positioned within the flexible inner shaft (“pusher”); in some examples (as shown in FIG. 1B) it may be outside of the inner shaft. In FIG. 1A, the column support may fit snugly within the flexible inner shaft (and may form a snug fit proximal to the inverted biopsy collector 110 (e.g., braid 110). The column support may reduce column buckling as the biopsy collector is pushed distally and everted from the device to collect material. The column support may be formed of any appropriate material, including Nitinol, a polymer, etc.

The biopsy collector 110 may include a first end 112 coupled to the distal end region of the flexible inner shaft 106 and a second end 114 coupled to the stepped distal end region 104 of the outer tubular member 102. In some examples, the first end 112 may be anchored or attached to an outer surface (and/or to a distal face) of the flexible inner shaft 106 and the second end 114 maybe anchored or attached to an outer surface (and/or to a distal face) of the stepped distal end region 104 (or to a distal face).

The stepped region may transition smoothly (e.g., in a continuous curve) over the first 1-20% of the length of the stepped region 104, or it may transition abruptly (as shown in FIGS. 1A-1B) as a step. The edge of the stepped region may be rounded.

As mentioned, the biopsy collector 110 may include and/or consist of a knit, braided, or woven material, such as a knit material formed of a wire (e.g., stainless steel, spring steel, Nitinol, etc.) to collect biopsy tissue. Alternatively or additionally, in some examples an additional material (e.g., a fabric or other material) may be formed as a sleeve, lining or other material that may be configured to collect and/or hold the biopsy material. The biopsy collector 110 may be an everting member that, as shown in FIG. 1B is either attached or includes a proximal portion that radially expands (e.g., is configured to expand radially when everted out of the stepped distal end region 104). In some examples a separate radially expanding sleeve portion is coaxial with or coupled along its length with the biopsy collector. In some examples the biopsy collector is formed of a knitted material that includes links that stack under compression (as in the un-inverted region when driven distally) to provide some column strength to push the distal face (where the biopsy collector is everting) distally into the lumen being biopsied.

In some examples, the flexible inner shaft 106 may be moved at a proximal end of the biopsy device 100. (For reference, the proximal end of the biopsy device 100 is toward the left of FIG. 1 and the distal end of the biopsy device 100 is toward the right side of FIG. 1). For example, movement of the flexible inner shaft 106 distally relative to the stepped distal end region 104, may extend the biopsy collector 110 distally out from the stepped distal end region 104. In some examples, the stepped distal end region 104 may also be moved concurrently (but in some cases at a different rate of speed or through a different distance) with the flexible inner shaft 106. Thus, the outer tubular member (e.g., the stepped distal end region 104) may assist the biopsy collector 110 in extending out of the biopsy device 100.

In some examples the biopsy collector 110 may be attached proximal to the distal end of the flexible inner shaft, so that as the flexible inner shaft is driven distally relative to the outer tubular member, the un-inverted biopsy collector 110 within the outer tubular member may be compressed, and in some examples may stack, while being driven distally. The more proximal end of the biopsy collector 110 may then be held in place by the outer wall of the flexible inner shaft, preventing it from buckling at least over the distal end region of the flexible outer shaft.

The flexible inner shaft 106 may be configured as a catheter having a central lumen, and a column support 108 may be included to provide lateral (radial) support for the biopsy collector 110. For example, a column support 108 may be slidably disposed within the flexible inner shaft so that it can be advanced as the biopsy collector 110 is everted and advanced, providing support and preventing kinking or buckling. The handle may coordinate advancing of the column support and the flexible inner member, which may be advanced at different rates to maintain the column support within the biopsy collector 110 as it is advanced (and in some cases, so that it does not extend far outside of the biopsy collector 110). In some examples the column support may be formed integral to and as part of the flexible inner shaft 106, as mentioned above (e.g., so that the biopsy collector 110 is attached at one end to a more proximal end of the flexible inner shaft). The column support 108 may be formed separately from, and attached to, the flexible inner shaft 106. In general, the column support 108 may have a smaller outer diameter than the flexible inner shaft 106 and may support the biopsy collector 110 within the stepped distal end region 104 and/or outside of the stepped distal end region. The column support 108 may reduce buckling of the biopsy collector 110 during deployment by providing an inner support to the biopsy collector 110.

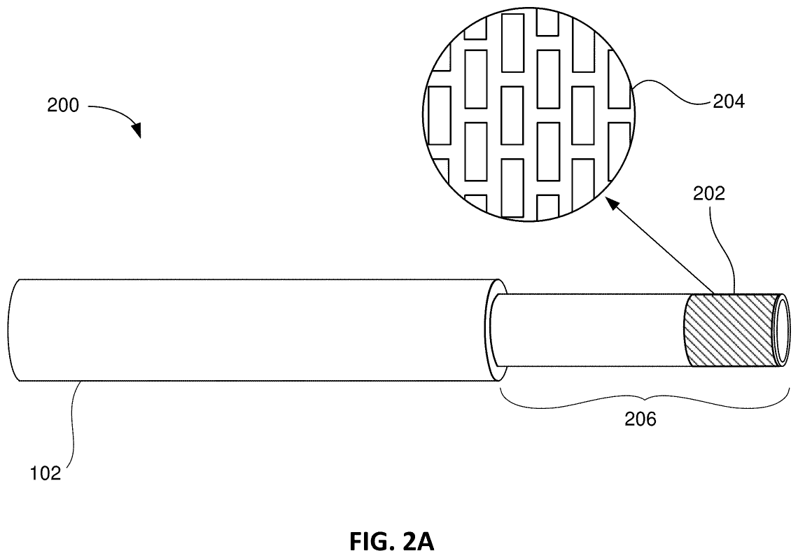

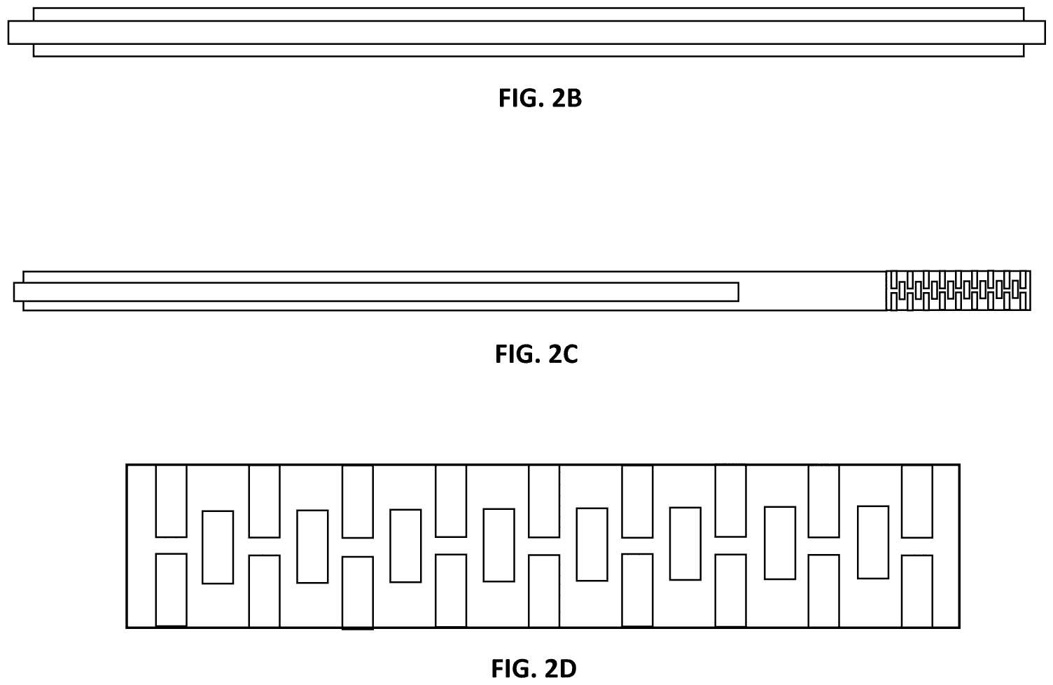

FIG. 2B shows an example of an internal support (configured here as a Nitinol rod, solid in this example, having an outer diameter of between about 0.018″ and 0.035″). In some examples the internal support may be a nitinol or steel tube (e.g. having a wall thickness of between 0.004″-0.006″), which may allow, e.g., injection of a fluid. The column support 108 may be removed proximally to increase the flexibility of the flexible inner shaft. Thus, the column support may be withdrawn partially or completely proximally, a described. In some examples the column support may be formed integrally into the distal end of the flexible inner shaft 106. Thus, the flexible inner shaft 106 and the column support 108 may be a continuous piece of material.

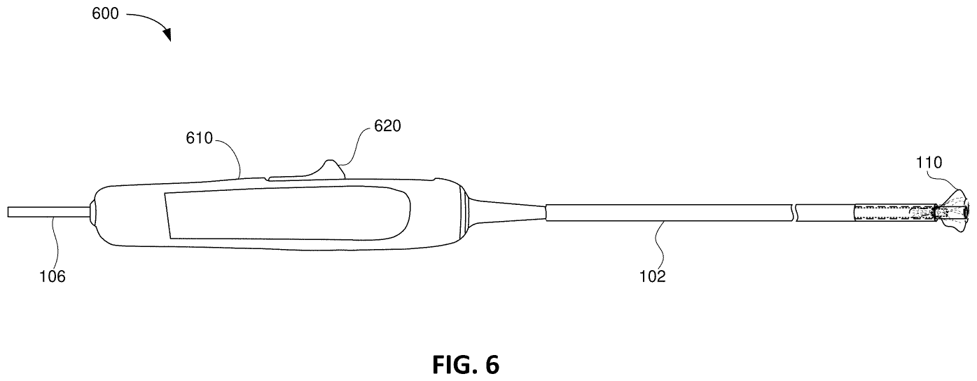

When the biopsy collector 110 is retracted into the stepped distal end region 104, the stepped distal end region 104 may protect the biopsy collector 110 and prevent contamination of the biopsy material as the biopsy device 100 is positioned within and/or withdrawn from the body. Any of these biopsy devices may also include a proximal handle as described in greater detail in reference to FIG. 6, below.

In general, either the outer tubular member (including in some examples the stepped distal end region) and/or the flexible inner member may include a flexible distal end region, including a flexible distal tip region that is configured to have a high column strength while still maintaining flexibility. For example, FIG. 2A shows an outer tubular member (or alternatively a flexible inner member) that is configured as described herein to include a flexible distal end region. In this example, the assembly 200 (e.g., an outer tubular member, including a stepped distal end region) includes a plurality of radially-oriented windows (arranged in an offset pattern) that may increase bending range without kinking or ovalization. In FIG. 2A, the example may be configured as an outer tubular member in which the stepped distal end region 104 may have an outer diameter slightly smaller than an inner diameter of the outer member 102. The stepped distal end region 104 in this example includes a distal region 202 that may be configured to enhance flexibility. In some examples, this region 202 may be selectively cut (e.g., laser cut) to form windows (as shown in close-up 204). Removal of the material may allow the region 202 to support a tighter bend radius than other regions that lack the removed material. View 204 shows one possible ablation pattern, however other patterns are contemplated. In some examples, material may be removed with a laser. In some other examples, the flexible region may be molded with reduced material within the region 202.

In the examples shown in FIGS. 2A-2D (when configured as an outer tubular member as shown in FIG. 1), the outer tubular member includes a stepped distal end region 104 that forms a step-down region 206 having a shoulder. The length of the step-down region 206 may be selected to provide particular advantages for anatomical regions (such as the entry into the fallopian tubes). In some examples, the step-down region 206 may be approximately 3 centimeters (e.g., about 1 cm, about 1.5 cm, about 2 cm, about 2.5 cm, about 3 cm, about 3.5 cm, about 4 cm, about 4.5 cm, about 5 cm, etc., such as between about 1.5 and 5 cm, between about 2 cm and 4.5 cm, etc.). The length of the step-down region 206 may enable the biopsy device 100 to be easily positioned into particular regions including, but not limited to fallopian tubes or other similar lumina having a narrowing (stricture) or bend.

In some examples, flexible distal end region may provide kink resistance, by allowing a high flexibility with a high compressive column force that may resist tensile stretch. For example, FIG. 2C schematically illustrates another example of a catheter (e.g., for the outer tubular member and/or for the flexible inner shaft) that includes a distal tip region with a high flexibility and a high column strength, similar to that shown in FIG. 2A. FIG. 2D shows an enlarged view of this flexible distal end region.

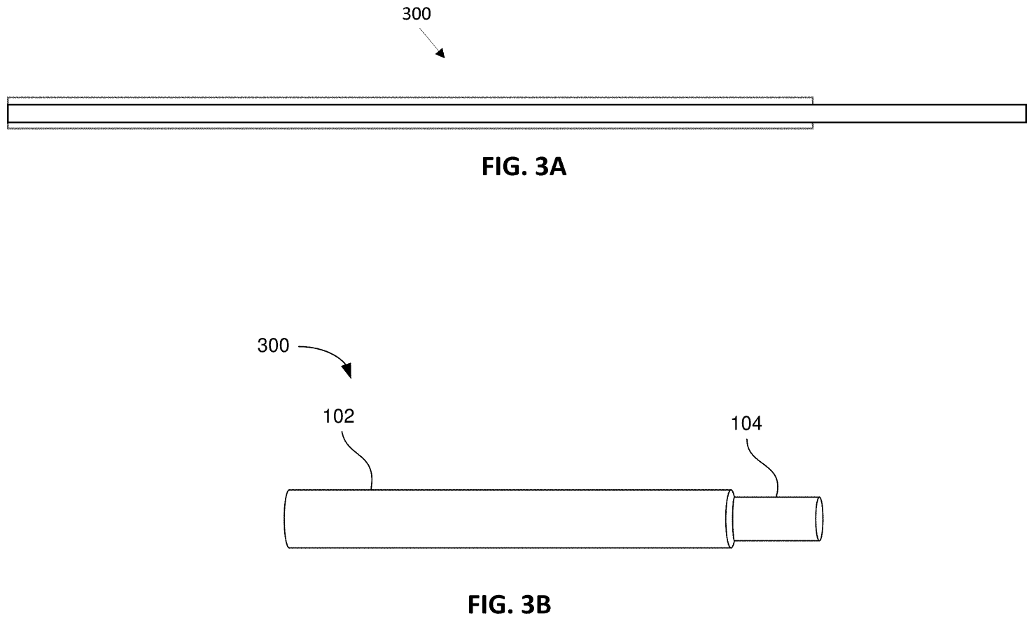

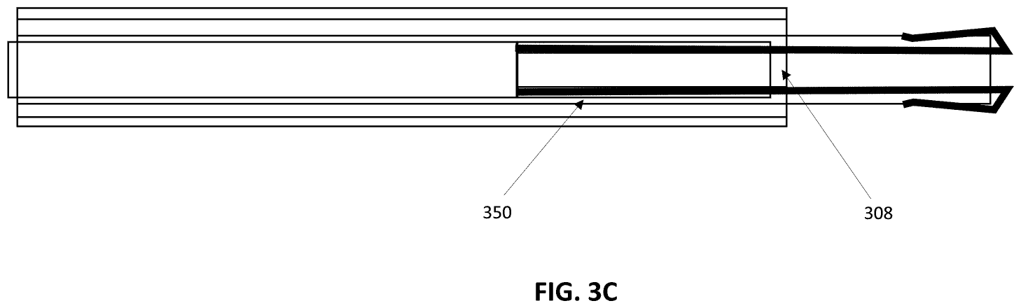

FIGS. 3A and 3B shows examples of a distal end of an outer tubular member (e.g., catheter) 300, as described herein. The outer tubular member may include a stepped distal end region 104 extending from an elongate outer tubular member body 102. As mentioned, the stepped distal end region may be fixed relative to the body of the outer tubular member. In some examples the length may be adjusted by sliding the internal body forming the stepped distal end region distally/proximally.

As shown, the outer diameter of the stepped distal end region has a smaller OD than the body of the outer tubular member. In this example, the stepped distal end region outer diameter may be between 50% and 80% of the OD of the body region of the outer tubular member (e.g., between about 60% and about 70%, about ⅔, etc.). As mentioned above, the distal tip region of the stepped distal end region may be flexible, as described for FIG. 2A.

Any of the apparatuses described herein may include an internal inverting member or column support element that wraps around the outer diameter to provide kink resistance at the flexible sleeve, as illustrated in FIG. 3C. In this example, the apparatus includes an outer sleeve 350 to resist braid expansion and/or braid buckling of the biopsy collector. This sleeve may squeeze the biopsy collector (e.g., the braid or knit forming the biopsy collector) to provide support when advancing. In FIG. 3C, additional support may be provided by a column support 308, which fits snugly into the inverted braid, so that as the braid is advanced distally, the outer wall of the column support prevents it from buckling (but may allow it to stack up and advance distally and evert). The column support may be Nitinol, as described above, and/or steel and/or polymer.

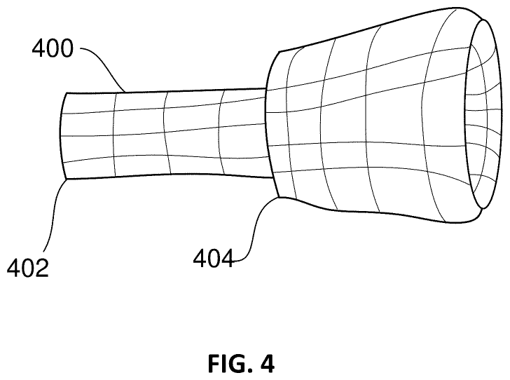

FIG. 4 shows one example of a biopsy collector 400 that may be used with any of the devices described herein. The biopsy collector 400 may be formed of a material that is knitted, woven, or braided. The biopsy collector 400 may include a first edge 402 and a second edge 404. The first edge 402 may be anchored to the flexible inner shaft 106 and the second edge 404 may be anchored to the distal end region (e.g., in some cases on the outer surface) of the stepped distal end region 104 (flexible inner shaft 106 and stepped distal end region 104 not shown for simplicity).

In general, the biopsy collector 110 may be configured so that, when pushed distally by pushing the flexible inner shaft 106 distally, the biopsy collector 110 may have sufficient column strength so that it is driven distally out of the distal end of the biopsy device 100. As mentioned, in some examples a support (e.g., column support) may be provided to prevent kinking or collapse. In some examples, the inner diameter of outer tubular member (e.g., of the stepped distal end region) may also provide support to the biopsy collector 110 and assist in pushing out (everting) the biopsy collector 110.

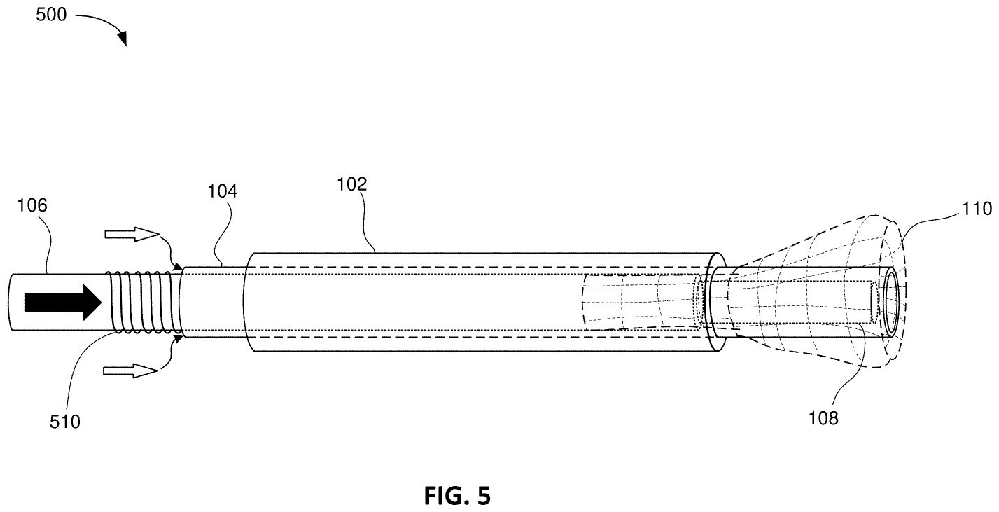

FIG. 5 shows another example of a biopsy device 500, as described herein. The biopsy device 500 may include the outer tubular member 102, having a stepped distal end region 104, a flexible inner shaft 106, a column support 108, and a biopsy collector 110. This example also includes a deployment bias 510 (configured as a spring). The deployment bias 510 may be configured to provide a force against the stepped distal end region 104 (denoted by the white arrows) to assist in everting the biopsy collector 110 from the stepped distal end region 104, e.g., by driving the flexible inner member distally, when the deployment bias is released. A releasable lock (not shown) may be used to release bias and advance the distal end. Note that the device shown in FIG. 5 is not to scale (for example, the length may be much longer). In some examples, the force from the bias 510 may drive the flexible inner member distally, pushing the everting biopsy collector 110 distally from one end (the distal end attached to the flexible inner member), while the proximal end is restrained, resulting in everting the distal face of the biopsy collector. The biopsy collector 110 may then contact cells and/or material and collect them from a lumen of the patient onto the biopsy collector.

As described, the biopsy collector 110 in this example has a first edge anchored to the flexible inner shaft 106 and a second edge coupled to the stepped distal end region 104. As the flexible inner shaft 106 receives a force (denoted by the black arrow), the flexible inner shaft 106 moves distally. Thus, the biopsy collector 110 is driven distally to evert. A reloading/return force that is opposite the deployment force (e.g., a force opposite to the black arrow) may be provided to retract the flexible inner shaft 106 and therefore to retract the biopsy collector 110 into the stepped distal end region 104. Alternatively, in some examples the apparatus may be configured so that a return force applied by the bias; the device may be manually extended and automatically retracted by releasing the bias (e.g., spring) to retract the flexible inner member and therefore the biopsy collector. In this case, tension from a bias 510 may assist in a smooth retraction of the biopsy collector 110 into the stepped distal end region 104. The force applied may be based on an associated spring constant. In some examples, the force from the spring 510 may be constant. In some other examples, the force from the spring 510 may be based on spring displacement. Any of these apparatuses may include a lock to prevent deployment of the bias until the lock is released, e.g., by a control on the handle.

FIG. 6 shows an example of a biopsy device 600, as described herein. The biopsy device 600 may include the outer member 102, the flexible inner shaft 106, biopsy collector 110, a handle 610, and a thumb slide 620. The outer tubular member 102 may also include the stepped distal end region 104 as depicted, but not shown here for simplicity. The device may also include a deployment (or retraction) bias that may be enclosed and supported within the handle 610. In the example shown in FIG. 6, the handle includes a control (e.g., thumb slide 620) that may be configured to selectively couple to the flexible inner shaft 106 to advance or retract it. Thus, a user may slide the thumb slide 620 to move the flexible inner shaft 106 distally to deploy the biopsy collector 110 and move the flexible inner shaft 106 proximally to retract the biopsy collector 110. In some examples, as the thumb slide 620 moves either distally or proximally, the flexible inner shaft 106 may be engaged with the thumb slide 620 to move an equivalent distance. In still other examples, the thumb slide 620 may engage with a ratchet (not shown for simplicity) to advance or retract the flexible inner shaft 106.

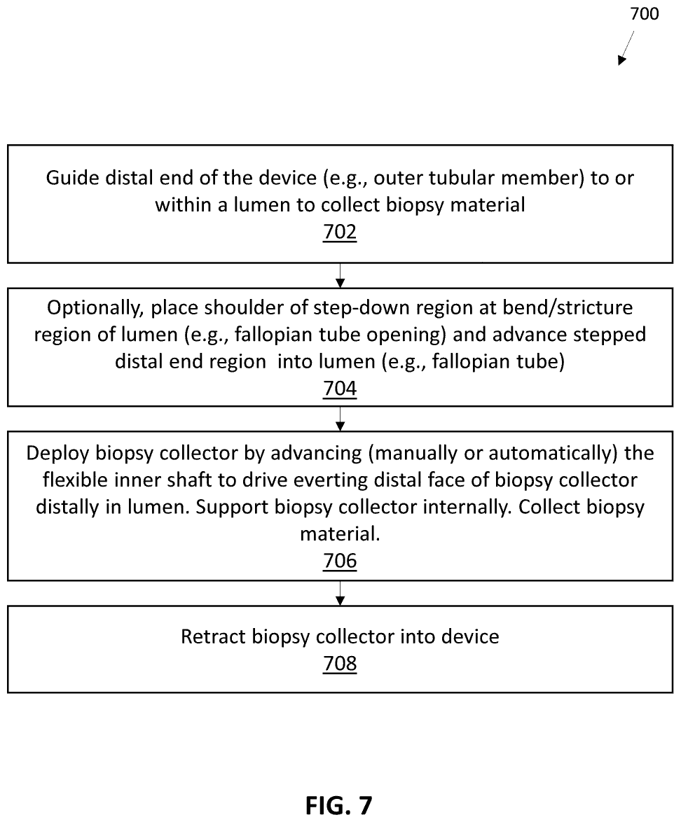

FIG. 7 is a flowchart depicting an example of one method 700 for performing a luminal biopsy using a biopsy device. Some examples may perform the operations described herein with additional operations, fewer operations, operations in a different order, operations in parallel, and some operations differently.

In FIG. 7, the method 700 may begin by guiding the distal end of the device through the body (e.g., minimally invasively, non-invasively (e.g., through natural orifice) to position the outer tubular member within the lumen 702. In some examples the distal end of the outer tubular member of the device is configured as a stepped distal end region. In some examples, the device may be advanced distally so that the stepped distal end region is positioned within the lumen until the shoulder formed by the stepped distal end region engages with a bend or stricture of the anatomy 704. For example, when inserting the device into the fallopian tube, the shoulder region may engage with the opening into the fallopian tube at the proximal tubal opening (also called the proximal ostium or os). The length of the stepped distal end region may be selected to position the distal opening of the device (out of which the biopsy collector may be extended) may be, e.g., 3 cm into the fallopian tube.

The position may be verified by, e.g., imaging (ultrasound, fluoroscopy, etc.). Once in position, the biopsy collector may be deployed by advancing, either manually or automatically (e.g., using a deployment bias), the inner flexible member to drive the biopsy collector so that it everts at a distal face that is advancing while everting, into the lumen 706. The inner flexible member may be drive by a control on the handle, and/or by releasing a lock on the handle, as described above. In some examples the biopsy collector may be supported (e.g., by a column support) as it is advanced, to prevent it from collapsing or kinking.

Once the material has been collected against the biopsy collector, it may be retracted, either manually or automatically (e.g., using a retraction bias) into the outer tubular member (e.g., in some examples into a stepped distal end region) 708.

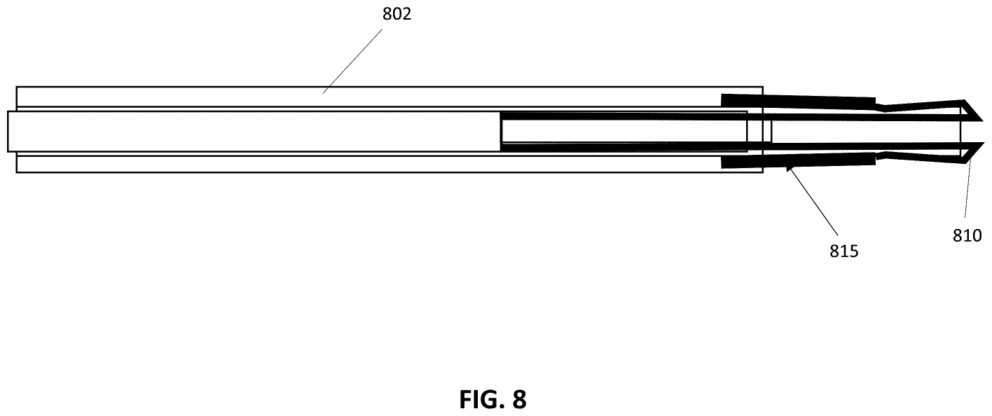

Any of the apparatuses described herein may also be configured to apply a radially outward force within the lumen as or before the biopsy collection is extended distally out of the apparatus. For example, FIG. 8 illustrates one example in which an everting braid region 815 at the proximal end of the biopsy collection can extend along the exterior of the distal end of the device to provide further kink resistance and strength to the tip of the flexible inner member. This region 815 may be a sleeve that is coupled to the outer tubular member 802 (including in some examples the stepped distal end region) at one end, and at the other end may connect to (or be continuous with) the biopsy collector 810, as shown in FIG. 8.

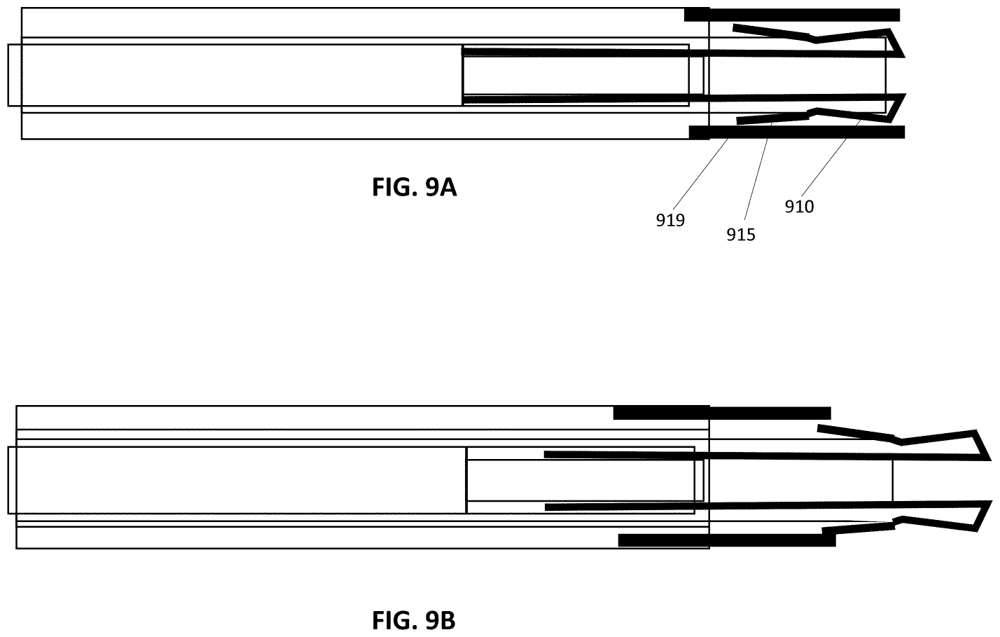

In some examples this sleeve (e.g., everting braid region) may be formed of a material that is shape-set to open radially (e.g., expand) outward to open the channel within the lumen which may make deployment of the biopsy collector more robust. In some examples this sleeve region may be configured to slide along the outer shaft to help advance the device into/out of the lumen being biopsied. For example, FIGS. 9A-9B illustrate an example in which an outer sleeve 919 may be driven to expand by an expandable portion 915 of the biopsy collector 910, to allow for easier deployment and depth penetration. This example may be configured as a self-sliding sheath, as shown in FIGS. 9A-9B.

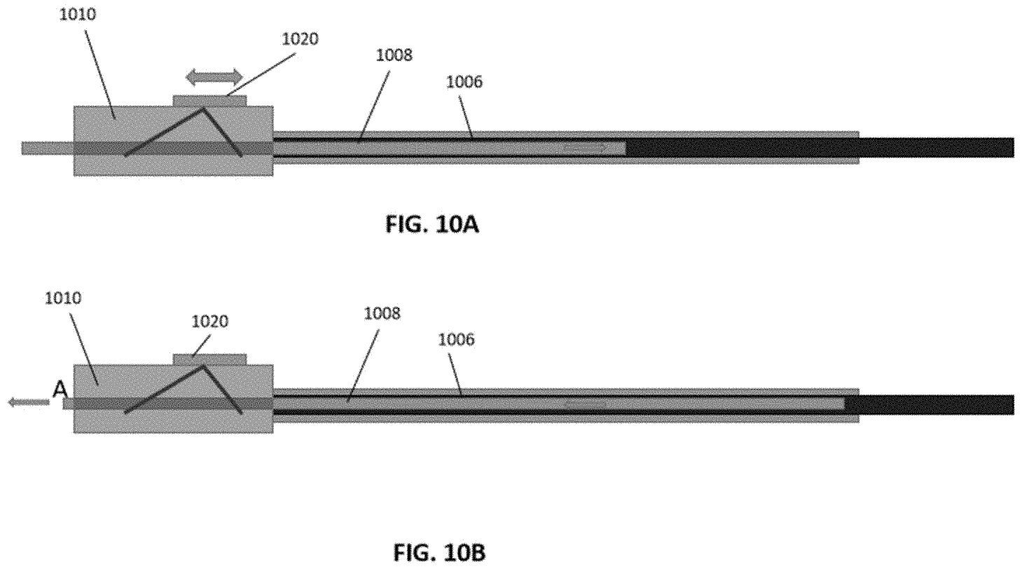

FIGS. 10A-10B illustrate the ratcheting handle mentioned above. In some examples the apparatus may be configured to include a ratcheting handle with or without a quick release (e.g., bias) that allows the device to be deployed in steps and returned in a single motion. For example, as shown schematically in FIG. 10, the device may include a ratcheting advance in the handle 1010 so that actuating of the control (e.g., thumb slider/trigger) 1020 advanced the biopsy collector (not shown) distally out of the device by one unit length. The until length may be arbitrarily determined (e.g., 0.5 mm, 1 mm, 2 mm, 3 mm, 4 mm, 5 mm, etc.). The control may then be reactivated to advance another unit. For example, in FIG. 10A, the device may be advanced in a stepwise manner by individual units by advancing the thumb slider 1020 distally to advance the biopsy collector by one unit (e.g., pushing the flexible inner member 1006 and/or support member 1008 distally) then sliding the thumb slider back to reset the ratchet (e.g., a pawl and/or gearing in the handle) so that it may again be advanced distally to advance the biopsy collector distally by another unit. The handle may therefor include the control mechanism to drive the push and/or support distally by one unit with each activation. In some examples, the handle may include a control to toggle from a stepwise ratcheting movement to a continuous distal advancement, so that the user may select between these two options.

As described above, the apparatus may be configured to allow retracting of the biopsy collector in a single movement of the control (e.g., finger slider or a second control). For example the inner member (and/or the support member) may be pulled back/returned in a single pull movement by the control. In some examples this retraction movement may be biased (e.g., by a spring, etc.) to occur automatically upon activation of a release (this may be referred to as a biased return), as shown in FIG. 10B.

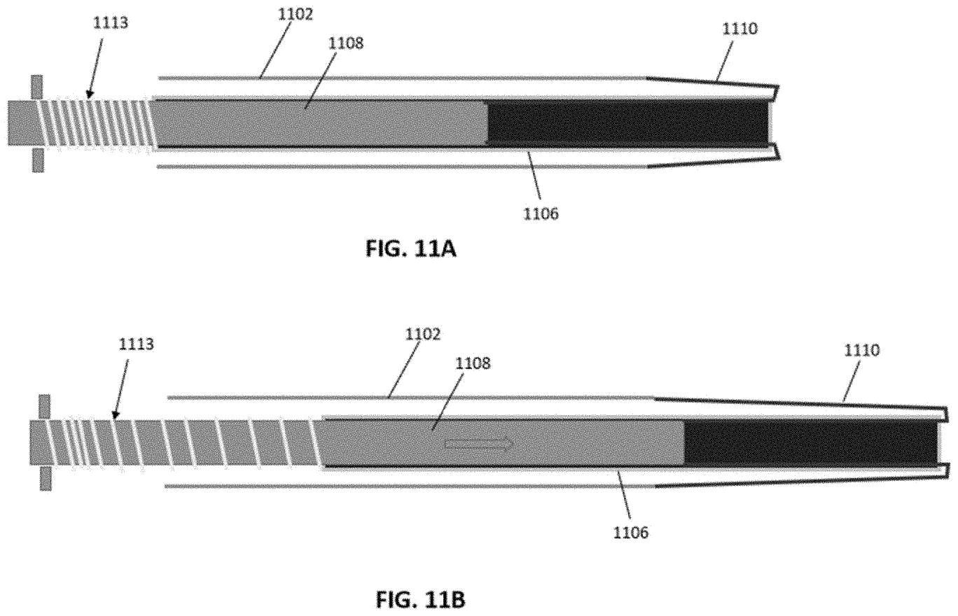

In some examples the apparatus may be configured to provide a biased advancement of the everting biopsy collector 1110 (e.g., braid). For example, FIGS. 11A-11B schematically illustrate a spring ratchet column delivery configuration in which a bias 1113 (e.g. compression spring) is coupled to the column support 1108 (and/or in some examples, the inner member/flexible inner shaft 1106). The compression spring may push the inner member forward, as shown in FIG. 11B, to support the everting biopsy collector 1110, driving (e.g., dragging) it out of the inner catheter as the inner catheter advances distally.

In any of these apparatuses, the biopsy collector 1110 may be held in a collapsed and/or constrained configuration within the flexible inner member prior to deployment, which may protect it from contamination and/or mechanical harm. Deploying the device may therefore pull the biopsy collector out of the flexible inner member 1106 so that it may be deployed, and in some cases may expand to contact the wall(s) of the lumen.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A biopsy device for taking a biopsy of a lumen, the device comprising:
an outer tubular member having an elongate body and a stepped distal end region, wherein the stepped distal end region has an outer diameter that is between 50% to 80% of an outer diameter of the elongate body and extends between 2-5 cm;
a flexible inner shaft arranged coaxially and extending within the outer tubular member; and
a biopsy collector having an everted tubular body, the biopsy collector coupled at a first end to a first distal end region to the flexible inner shaft, wherein the biopsy collector everts over itself at a distal face so that a second end of the biopsy collector is coupled to the stepped distal end region;
wherein the flexible inner shaft is configured to move axially within the outer tubular member to drive the distal face of the biopsy collector distally as the flexible inner shaft is moved distally relative to the outer tubular member.

2. The biopsy device of claim 1, further comprising a column support extending within a lumen of the biopsy collector, and wherein the biopsy collector is configured to support against an outer surface of the column support as the flexible inner shaft is driven distally.

3. The biopsy device if claim 2, wherein the column support comprises a rod that is configured to slide axially within a lumen of the flexible inner shaft.

4. The biopsy device of claim 1, further comprising a handle at a proximal end of the outer tubular member.

5. The biopsy device of claim 1, further comprising a deployment bias coupled to the flexible inner shaft and configured to drive the flexible inner shaft distally to deploy the biopsy collector distally when a lock restraining the deployment bias is released.

6. The biopsy device of claim 4, further comprising a thumb slide configured to move the flexible inner shaft relative to the outer tubular member.

7. The biopsy device of claim 1, wherein the stepped distal end region is coaxially arranged within an outer tubular member, such that the stepped distal end region may move axially in a distal and proximal direction relative to the outer tubular member.

8. The biopsy device of claim 1, further comprising a sleeve configured to expand radially from a distal end of the device when the biopsy collector is advanced distally.

9. The biopsy device of claim 1, wherein the biopsy collector comprises a plurality of knitted strands.

10. The biopsy device of claim 1, wherein the biopsy collector comprises a braid, a knit or a weave.

11. The biopsy device of claim 1, wherein the stepped distal end region comprises a flexible region comprising a plurality of cut-out regions radially arranged around a circumference of the stepped distal end region that are configured to increase flexibility without reducing compressive column strength.

12. A biopsy device for taking a biopsy of a lumen, the device comprising:
an outer tubular member having an elongate body and a stepped distal end region;
a flexible inner shaft arranged coaxially within the outer tubular member;
a biopsy collector having an everted tubular body, the biopsy collector coupled at a first end to the flexible inner shaft and a second end to the outer tubular member, wherein, in an undeployed state, the biopsy collector is longitudinally compressed within a lumen of the flexible inner shaft, further wherein the biopsy collect everts over itself at a distal face; and
a column support within the flexible inner shaft;
wherein the flexible inner shaft is configured to move axially distally within the outer tubular member to deploy the biopsy collection distally from the outer tubular member so that it everts over itself as the flexible inner shaft is moved distally relative to the outer tubular member.

13. A biopsy device for taking a biopsy of a lumen, the device comprising:
an outer tubular member having an elongate body and a stepped distal end region, wherein the stepped distal end region has an outer diameter that is between 50% to 80% of the outer diameter of the elongate body and wherein the stepped distal end region extends between 2 cm and 5 cm;
a flexible inner shaft arranged coaxially extending within the outer tubular member;
a biopsy collector having an everted tubular body, the biopsy collector coupled at a first end to the flexible inner shaft, wherein the biopsy collect everts over itself at a distal face so that a second end of the biopsy collector is coupled to the stepped distal end region; and
a column support within the biopsy collector and/or the flexible inner shaft;
wherein the flexible inner shaft is configured to move axially distally within the outer tubular member to drive the distal face of the biopsy collector distally as the flexible inner shaft is moved distally relative to the outer tubular member.

14. A method of collecting biopsy material from within a lumen, the method comprising:
guiding an outer tubular member of a biopsy device to a lumen;
advancing a stepped distal end region of the outer tubular member into the lumen until a shoulder region of the outer tubular member is against or adjacent to an opening into the lumen or a bend in the lumen;
advancing a flexible inner shaft of the device distally relative to the outer tubular member to evert a tubular and flexible biopsy collector over itself as the biopsy collector extends distally, wherein a distal face of the biopsy collector advances distally as the flexible inner shaft is advanced distally;
collecting biopsy material onto an inverted outer face of the biopsy collector within the lumen; and
retracting the flexible inner shaft proximally to invert the inverted outer face of the biopsy collector back into the outer tubular member.

15. The method of claim 14, wherein advancing the flexible inner shaft of the device distally relative to the outer tubular member to evert the biopsy collector over itself comprises pulling the biopsy collector out of the flexible inner shaft.

16. The method of claim 14, wherein advancing the flexible inner shaft comprises releasing a lock to automatically advance the flexible inner shaft of the device distally relative to the outer tubular member.

17. The method of claim 15, wherein releasing comprises releasing a deployment bias.

18. The method of claim 14, further comprising supporting the tubular and flexible biopsy collector on a column support within the tubular and flexible biopsy collector as the flexible inner shaft is advanced.

19. The method of claim 14, further comprising expanding a sleeve radially from a distal end of the outer tubular member while advancing the flexible inner shaft of the device distally.

20. The method of claim 14 wherein advancing the flexible inner shaft comprises operating a thumb slide on a handle of the biopsy device.

21. The method of claim 14, wherein guiding the outer tubular member of the biopsy device to the lumen comprises positioning the outer tubular member at the opening to a fallopian tube.

22. A method of collecting biopsy material from within a lumen, the method comprising:
guiding an outer tubular member of a biopsy device to a lumen;
advancing a stepped distal end region of the outer tubular member into the lumen until a shoulder region of the outer tubular member is positioned against an opening into the lumen or a bend in the lumen;
advancing a flexible inner shaft of the device distally relative to the outer tubular member to evert a tubular and flexible biopsy collector over itself as the biopsy collector extends out of the flexible inner shaft, wherein a distal face of the biopsy collector advances distally as the flexible inner shaft is advanced distally;
collecting biopsy material onto an inverted outer face of the biopsy collector within the lumen; and
retracting the flexible inner shaft proximally to invert the inverted outer face of the biopsy collector back into the outer tubular member.
